(19) United States
(12) Patent Application Publication
(10) Pub. No.: US 2011/0076756 A1
(43) Pub. Date: Mar. 31, 2011

(54) VARIABLE VOLUME BIOREACTOR

(76) Inventor: Ian Malcolm Wright, Queensland (AU)

(21) Appl. No.: 12/524,064

(22) PCT Filed: Jan. 22, 2008
(86) PCT No.: PCT/AU2008/000075

§ 371 Date: Nov. 22, 2010

(30) Foreign Application Priority Data
Jan. 22, 2007 (AU) 2007900291
Oct. 26, 2007 (AU) 2007905929

(51) Int. Cl. C12M 1/00

(52) U.S. Cl. 435/292.1

(57) ABSTRACT

A variable volume bioreactor including a rigid core associated with a light source, an outer expandable growth containment portion located concentrically about the rigid core, a lower end portion associated with an inlet to supply culture medium and an outlet, and an upper end cap with an opening therein through which the rigid core passes, wherein the outer containment portion expands as the cellular biological material contained in said portion grows and expands.

### FIELD OF THE INVENTION

The present invention relates to bioreactors and particularly to low cost, reusable bioreactors.

### BACKGROUND ART

For many years, bioreactors have been considered to be an ideal technical solution for the growing of feedstock for the extraction of oils. Bioreactors have also been suggested as a method to remove carbon dioxide from industrial emissions. The practical use of bioreactors has had limited success due to the cost of building and maintaining the bioreactor vessels and also the difficulty in removing the products from the reactor vessel once mature.

Bioreactors degrade contaminants in water with microorganisms through attached or suspended biological systems. In suspended growth systems, such as activated sludge, fluidized beds, or sequencing batch reactors, contaminated ground water is circulated in an aeration basin where a microbial population aerobically degrades organic matter and produces CO₂, H₂O, and new cells. The cells form a sludge, which is settled out in a clarifier, and is either recycled to the aeration basin or disposed. In attached growth systems, such as upflow fixed film bioreactors, rotating biological contactors (RBCs), and trickling filters, microorganisms are established on an inert support matrix to aerobically degrade water contaminants.

The two types of devices providing for variable volume during cell culture without compromising sterility of the culture that have been described, moreover, present significant restrictions that hinder their application. Several forms of a chamber for cell culture based on a bag, which conceptually could allow variable volumes for cultures, have been described previously (e.g., U.S. Pat. Nos. 5,686,304 and 5,714,384), but the flexible walls present in these and other bags do not provide tight control of volumes, do not provide rigid surfaces for culture of adherent cells, nor present chambers with well-defined geometries for well-defined perfusions (e.g., uniform hydrodynamic shear stresses required for many adherent cells). Deformability of a wall of a chamber in general (e.g., as practiced by U.S. Pat. No. 6,152,163), leads to these inherent limitations. Alternatively, U.S. Pat. No. 5,707,868 describes the use of a piston-based design as a variable-volume chamber for cell culture. This type of design, similar in concept to other piston-based designs for biotechnological applications described in U.S. Pat. Nos. 5,143,847, 6,007,472, and 6,290,910, are cumbersome mechanically and not well-suited to large, planar cultures of adherent monolayers.

The present invention has been specifically devised in order to provide a low-cost bioreactor with simple, effective design capable of providing a continuous or reusable process.

It will be clearly understood that, if a prior art publication is referred to herein, this reference does not constitute an admission that the publication forms part of the common general knowledge in the art in Australia or in any other country.

### SUMMARY OF THE INVENTION

The present invention is directed to a bioreactor, which may at least partially overcome at least one of the abovementioned disadvantages or provide the consumer with a useful or commercial choice.

With the foregoing in view, the present invention in one form, resides broadly in a bioreactor including a rigid core associated with a light source, an outer expandable growth containment portion located concentrically about the rigid core, a lower end portion associated with an inlet to supply culture medium and an outlet, and an upper end cap with an opening therein through which the rigid core passes, wherein the outer containment portion expands as the cellular biological material contained in said portion grows and expands.

The present invention therefore is intended to provided a device for culturing cellular biological material in the form of solid particles in which the said solid particles are brought into contact with a liquid culture medium, making it possible to keep the density by volume of the said cellular biological material substantially constant with respect to the volume of the said culture medium.

It has been found that it is advantageous to carry out culturing at a constant density by volume of cells in order to have correct development of the embryos or even to reduce this density by increasing the volume of the culturing enclosure, partly due to the release of certain compounds during maturation of the embryos, it being possible for these compounds to have a stimulating or inhibiting effect on the maturation.

The bioreactor of the present invention can be adapted for any use, but is particularly well-suited for growing carbon dioxide sequestrating biological material, methane digestion, fuel production or water production. Any biological material or growth culture medium may be used.

The bioreactor of the present invention may be of any shape, but a cylindrical shape is preferred. Typically, the components of the bioreactor will be located concentrically about a central axis. The bioreactor will normally be substantially vertical in orientation although other orientations may be used. In the vertical orientation, growth of the biological material will force expansion of the containment portion upwardly.

The bioreactor of the present invention will normally include a liquid culture medium. The biological material will normally be referred to as “solid particles” in order to distinguish them from the liquid culture medium only.

The bioreactor will of course be provided in differing sizes depending upon application, but preferred sizes are between 1 and 10 meters in height with a particularly preferred configuration of approximately 2 m in overall height (or length) and between 20 cm to 5 m in diameter with a particularly preferred configuration of approximately 20 cm to 1 m in diameter.

The bioreactor of the present invention also includes a rigid core associated with a light source. The rigid core will typically be oriented substantially vertically. The core is preferably formed of a hollow tubular member which will also preferably be cylindrical.

The core is preferably manufactured of a translucent plastic such as translucent PVC but other materials for example, strengthened glass may be used. The core is also preferably longer (higher) than the length (height) of a containment portion expanded to maximum expansion.

The core is preferably maintained substantially vertically by a support frame or an overhead fixing frame. The fixing frame is preferably attached to the core at an upper portion. The attachment between the core and the fixing frame is preferably removable in order to change or repair the light source, for example.

The rigid core typically extends from adjacent the lower end cap, upwardly. The core is typically provided with a closed bottom and an open top.

The core is preferably provided with an attachment portion on a lower portion and normally on an outer surface of the closed bottom of the core.

The dimensions of the core may vary, but for example, for a 6 m high bioreactor, the core is preferably approximately 10 cm in diameter. The dimensions of the core may be as low as 5-10 mm if a fibre optic light source is used.

The rigid core is associated with a light source preferably a controlled light source to provide optimum conditions for growth of the biological material. Any suitable light source may be used. Examples include fluorescent light or a fiber-optic array. The light source may be controlled to provide a particular wavelength or band of light.

The bioreactor of the present invention includes an outer expandable growth containment portion located concentrically about the rigid core. The containment portion may include an outer wall and an inner wall with the containment zone defined therebetween. The inner wall is preferably located adjacent the core and the outer wall is then spaced concentrically outwardly therefrom.

The inner wall of the containment portion is preferably translucent and have a concertina-like configuration allowing length (height) adjustment. The outer wall of the containment portion is preferably opaque and have a concertina-like configuration allowing length (height) adjustment.

Each of the concertina-like walls will preferably be formed of a flexible material with a plurality of hinge or fold lines extending circumferentially about the wall to allow expansion and contraction. The portions of each wall located between the hinge or fold lines will typically be adapted to maintain a substantially planar shape and resist deformation or bulging of the wall under load. The hinge lines may therefore be the main functional component in allowing the expansion and contraction.

The preferred material of construction for each of the walls is plastic with the type of plastic chosen to suit the requirements of each of the walls.

The inner wall may be provide with a closed lower end. The upper end of the inner wall will typically be attached relative to the upper end cap in order to allow the inner wall to be drawn upwardly as the upper cap moves upwardly. An upper end or portion of the inner wall may therefore be provided with an externally threaded portion.

Both walls will typically be provided with the optimum surface conditions for growth of biological material and material and/or physical characteristics adapted to suit.

The inner wall is also preferably further provided with an attachment portion adapted to attach to the rigid core attachment portion so that both can be removed from within the outer wall together if required.

The bioreactor of the present invention includes a lower end portion associated with an inlet to supply culture medium when required, and an outlet. The lower end portion may be a cap, typically a support member that supports the remainder of the bioreactor components. The lower end cap is suitably attached to the outer wall of the containment portion and is preferably sealed thereto, typically to an outer surface of the outer wall.

The lower end cap is normally sized to define the outer dimension of the expanded reactor.

The inlet and outlet are preferably in fluid communication with the containment portion. Both the inlet and the outlet are preferably selectively operable and valve assemblies will normally be provided in association with each. The inlet and outlet are typically horizontally opposed to one another. The inlet is typically smaller in diameter than the outlet.

The bioreactor of the present invention includes an upper end cap with an opening therein through which the rigid core passes. The upper end cap is preferably attached to the outer wall in a manner similar to the lower end cap. The upper end cap will normally be formed of a material similar to that used to form the lower end cap namely a metal or more typically, a plastic material.

The upper end cap usually attaches to the inner wall of the containment portion. The opening in the upper end cap for the rigid core is normally located centrally with a secondary opening provided for a bleed/pressure relief valve. The secondary opening is normally threaded to provide a removable attachment means for the bleed/pressure relief valve.

The central opening may have an associated collar to attach the inner wall of the containment portion thereto. The collar will preferably be annular to receive the rigid core and then extend through the central opening in the upper end cap which will be suitably dimensioned to receive both the rigid core and the collar.

The collar will normally be provided with an outer seating portion to locate the collar on the end cap and be engaged there. An O-ring or similar sealing means will also be provided on the collar in an internal seating groove to form a fluid tight seal with the outer surface of the rigid core. The lower portion of the collar will be provided with an internally threaded portion engageable with the externally threaded portion on the inner wall of the containment portion.

In an alternative embodiment, the containment portion may be provided between the outer containment wall and the light source, without an inner wall. According to this configuration, the movable end cap (typically the upper end cap) will normally be provided with a sealing grommet associated with the opening in the end. The sealing grommet preferably closely receives the light source and is slidable upwardly and downwardly relative to the light source. The sliding action will also assist with keeping the light source clean.

In use, an initiating portion of a chosen biological material is placed or fed into the containment portion of the bioreactor, together with a suitable initial charge of culture medium. The conditions within the containment portion are thereafter controlled to optimise growth in the bioreactor. As the material grows the containment portion expands. Once mature, the rigid core and inner wall can be removed as can the mature material, and the containment portion can either be cleansed and collapsed ready for re-use or the mature material can simply be removed, and a new culture medium inserted, with the remnants of the mature material providing the starter biological material for the new batch. The remnants will typically be trapped during compression or collapse of the containment portion between the concertina parts of the wall(s).

The outer wall of a particularly preferred embodiment may be additionally supported through the provision of one or more reinforcement members. The reinforcement members will normally act to support the flexible material used as the outer wall in order to assist with maintaining the shape of the outer wall.

Various configurations and numbers of reinforcement members may be used. For example, according to one preferred embodiment, a plurality of annular or ring-shaped reinforcement members can be provided, spaced over the height of the outer wall with a portion of the ring supporting the outer wall. The reinforcement members can be provided either on the inside of the outer wall on the outside of the outer wall.

Where the reinforcement members of this embodiment are provided internally, the reinforcement members may be provided in a portion of the outer wall which bulges outwardly, and where the reinforcement members of this embodiment are provided externally of the outer wall, the ring members will typically be provided in portions of the outer wall between outward bulges.

In an alternate embodiment, a helical reinforcement member may be provided within (or outside) the outer wall. Provision of a helical reinforcement member is advantageous in the as well as supporting the outer wall during expansion, the helical coil is also self aligning when of the outer wall is compressed.

In a further preferred embodiment, the reinforcement members may be integrated into the outer wall and formed therewith. In this form, the reinforcement members may preferably be a portion of the wall with increased thickness. Normally, the reinforcement members/portions of this embodiment may be provided as an outwardly bulging part of the outer wall. According to this embodiment, the outer wall may be manufactured by extrusion of a particular thickness of wall portion and then stretching a portion of that wall to decreased thickness leaving thickened reinforcement portions interspersed with the thinner wall portions.

According to further embodiments of the invention, a plurality of bioreactors may be provided mounted on a base member. The base members may form a part of a fixed assembly or a portable assembly.

The base member will typically be provided with utilities such as heating and a biological material inlet and outlet with appropriate connections to each bioreactor. Each bioreactor is typically attached to the base through the provision of a base fitting and attaching the outer wall of the bioreactor to the base fitting. Typically, the connection between the base fitting and the bioreactor will be formed using a clamping means.

Preferably, the base member will be a structural member capable of being lifted by lifting means such as a forklift. The base member will preferably be provided with a biological material feed inlet and a biological material outlet with appropriate connections to each of the bioreactor is mounted on the base member. The base member will also preferably include a conduit for a heating medium with an inlet and an outlet and a conduit in communication with each of the bioreactors.

The base member is preferably configured so that more than one base member and can be located adjacent one another and the heating medium inlet and outlet as well as the biological material inlet and outlet of adjacent base members are aligned or communicate. This will allow a single pump means to move heating medium through all of the base members and another single pump means to move on biological material through all of the base members. Preferably, the biological material conduit and the heating medium conduit are co-current.

The inlets and outlets of the base member will preferably be associated with valve means.

The present invention is particularly adapted to use salt water or brine based bacteria.

Using appropriate bacteria, the inventor has found that a single bioreactor with a maximum height of approximately 2 m and approximately 30 cm in diameter can absorb up to 2.75 kg in a 24 hour period of carbon dioxide.

According to an alternative embodiment, there may be a secondary chamber provided surrounding the bioreactor of the invention. The secondary chamber will typically be a process vessel and according to a preferred embodiment will also be a bioreactor. This embodiment of the invention is preferably adapted for use on portable or transportable equipment to treat particularly emissions from the equipment.

According to this embodiment, the secondary chamber will be a secondary bioreactor to treat the incoming material to remove the same or different pollutants as the inner, primary bioreactor. The inlet will preferably be into the secondary chamber and normally towards a lower portion of the secondary chamber. Located at or towards an upper portion of the secondary chamber is typically an outlet which is in turn connected to a lower inlet into the primary bioreactor.

The primary bioreactor and secondary bioreactor are preferably separated by a separating assembly which will normally be rigid. The separating assembly will normally have a substantially centrally located outlet which according to a preferred embodiment, is coaxially mounted with the light source.

Both bioreactors may be variable volume according to this embodiment or only the inner, primary bioreactor may be variable volume. Culture medium and growth medium will normally be provided in portion between the separating assembly and the secondary chamber wall. Oxygen supply apparatus may also be provided in a lower portion of the secondary bioreactor.

### BRIEF DESCRIPTION OF THE DRAWINGS

Various embodiments of the invention will be described with reference to the following drawings, in which:

FIG. 1 is a schematic view from the side of a bioreactor according to a preferred aspect of the present invention, in a partially expanded condition.

FIG. 2 is a schematic view from the side of the bioreactor illustrated in FIG. 1 in a compressed condition.

FIG. 3 is a detailed section view of an upper portion of the bioreactor illustrated in FIG. 1.

FIG. 4 is a sectional elevation view of a containment portion according to a preferred embodiment.

FIG. 5 is a sectional elevation view of a containment portion according to an alternative embodiment.

FIG. 6 is a sectional elevation view of a containment portion according to an alternative embodiment.

FIG. 7 is a sectional elevation view of a containment portion according to an alternative embodiment.

FIG. 8 is a sectional elevation view of a containment portion according to an alternative embodiment.

FIG. 9 is a sectional elevation view of a containment portion according to an alternative embodiment.

FIG. 10 is a view from above of a base mount for a cluster of four bioreactors with attendant utilities according to a preferred embodiment.

FIG. 11 is a side elevation view of the base mount of FIG. 10 with containment portions attached.

FIG. 12 is a sectional side elevation view of bioreactor cluster according to a first preferred embodiment, in a partially extended condition.

FIG. 13 is a view from above of a base mount for a cluster of four bioreactors with attendant utilities according to a preferred embodiment.

FIG. 14 is a side elevation view of the base mount of FIG. 13 with containment portions attached.

FIG. 15 is a sectional side elevation view of bioreactor cluster according to a first preferred embodiment, in a fully extended condition.

FIG. 16 is a view from above of a base mount for a cluster of four bioreactors with attendant utilities according to a preferred embodiment.

FIG. 17 is a side elevation view of the base mount of FIG. 16 with containment portions attached.

FIG. 18 is a sectional side elevation view of bioreactor cluster according to a second preferred embodiment, in a partially extended condition.

FIG. 19 is a view from above of a base mount for a cluster of four bioreactors with attendant utilities according to a preferred embodiment.

FIG. 20 is a side elevation view of the base mount of FIG. 19 with containment portions attached.

FIG. 21 is a sectional side elevation view of bioreactor cluster according to a second preferred embodiment, in a fully extended condition.

FIG. 22 is a view from above of a base mount for a cluster of four bioreactors with attendant utilities according to a preferred embodiment.

FIG. 23 is a side elevation view of the base mount of FIG. 22 with containment portions attached.

FIG. 24 is a sectional side elevation view of bioreactor cluster according to a third preferred embodiment, in a partially extended condition.

FIG. 25 is a view from above of a base mount for a cluster of four bioreactors with attendant utilities according to a preferred embodiment.

FIG. 26 is a side elevation view of the base mount of FIG. 25 with containment portions attached.

FIG. 27 is a sectional side elevation view of bioreactor cluster according to a third preferred embodiment, in a fully extended condition.

FIG. 28 is a view from above of a base mount for a cluster of four bioreactors with attendant utilities according to a preferred embodiment.

FIG. 29 is a side elevation view of the base mount of FIG. 28 with containment portions attached.

FIG. 30 is a sectional side elevation view of bioreactor cluster according to a fourth preferred embodiment, in a partially extended condition.

FIG. 31 is a view from above of a base mount for a cluster of four bioreactors with attendant utilities according to a preferred embodiment.

FIG. 32 is a detail sectional elevation view of a portion of a containment portion according to a fourth preferred embodiment.

FIG. 33 is a side elevation view of the base mount of FIG. 32 with containment portions attached.

FIG. 34 is a sectional side elevation view of a bioreactor cluster according to a fourth preferred embodiment, in a fully extended condition.

FIG. 35 is a sectional side view of a bioreactor assembly with an outer secondary bioreactor according to a preferred embodiment, with the primary bioreactor in partially extended condition.

FIG. 36 is a sectional side view of the bioreactor assembly illustrated in FIG. 35 with the primary bioreactor in a fully extended condition.

### DETAILED DESCRIPTION OF THE PREFERRED EMBODIMENT

According to a preferred aspect of the present invention, an expandable bioreactor is provided.

The bioreactor 10 of the illustrated embodiment includes a rigid core 11 associated with a light source 12, an outer expandable growth containment portion located concentrically about the rigid core 11, a lower end cap 13 with an inlet 14 to supply culture medium and an outlet 15, and an upper end cap 16 with an opening 17 therein through which the rigid core 11 passes.

The rigid core 11 of the illustrated embodiment is oriented substantially vertically. The core 11 is formed of a hollow tubular member which is cylindrical.

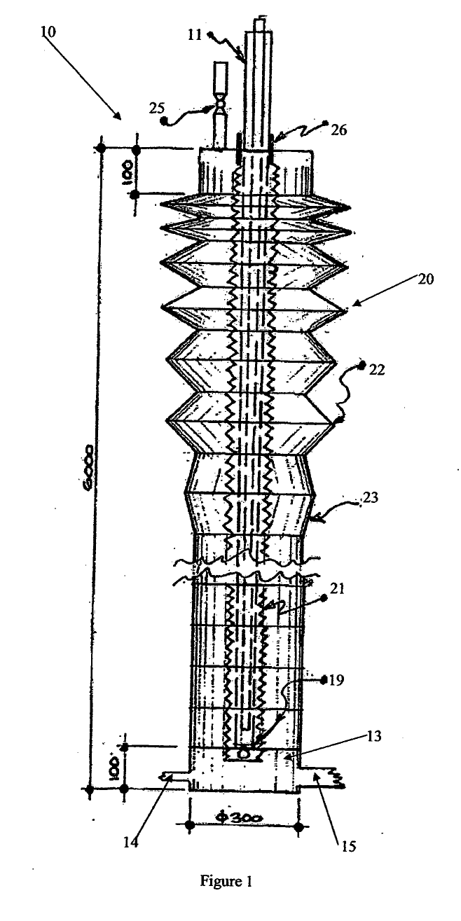

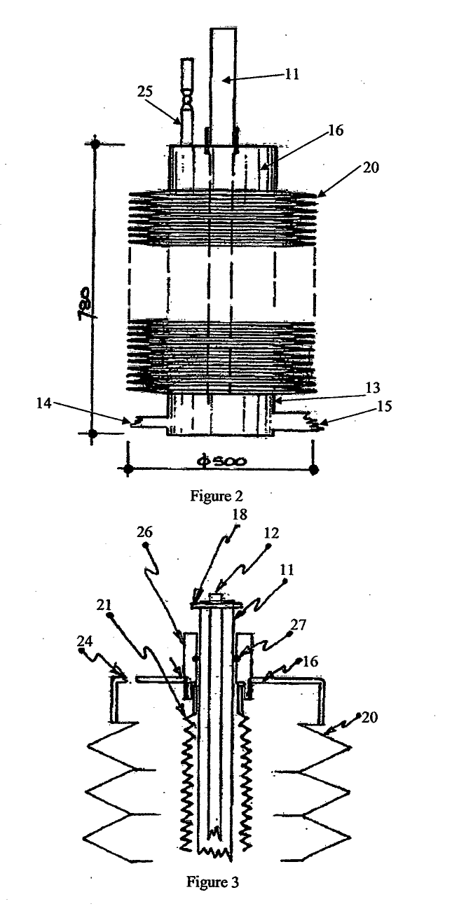

The core 11 is manufactured of a translucent plastic such as translucent PVC. As illustrated in FIG. 1 in particular, the core 11 is longer (higher) then the length (height) of a containment portion expended to maximum expansion.

The core 11 is maintained substantially vertically by an overhead fixing frame 18. The fixing frame 18 is attached to the core 11 at an upper end. The rigid core 11 extends from adjacent to the lower end cap 13 upwardly. The core is provided with a closed bottom and an open top.

The core 11 is also provided with an attachment portion 19 on an outer surface of the closed bottom of the core 11.

The rigid core 11 is associated with a light source 12 preferably a controlled light source to provide optimum conditions for growth of the biological material. According to the illustrated embodiment, a fluorescent light extending over the height of the containment portion is used.

The outer expandable growth containment portion is located concentrically about the rigid core. The containment portion includes an outer wall 20 and an inner wall 21 with the containment zone defined therebetween. The inner wall 21 is located adjacent the core 11 and the outer wall 20 is then spaced concentrically outwardly therefrom.

The inner wall 21 of the containment portion is translucent and has a concertina-like configuration allowing length (height) adjustment as does the outer wall of the containment portion, though the outer wall 20 is preferably opaque.

Each of the concertina-like walls is formed of a flexible material with a plurality of hinge or fold lines 22 extending circumferentially about the wall to allow expansion and contraction. The portions 23 of each wall located between the hinge or fold lines 22 are adapted to maintain a substantially planar shape and resist deformation or bulging of the wall.

The inner wall 21 is provided with a closed lower end. As illustrated best in FIG. 3, the upper end of the inner wall 21 is attached relative to the upper end cap 16 in order to allow the inner wall 21 to be drawn upwardly as the upper cap 16 moves upwardly. An upper portion of the inner wall 21 is provided with an externally threaded portion.

The inner wall 21 is also provided with an attachment portion adapted to attach to the rigid core 11 attachment portion 19 so that both can be removed from within the outer wall 20 together if required.

The lower end cap 13 is attached to the outer wall 20 of the containment portion and is sealed thereto. The lower end cap 13 is sized to define the outer dimension of the expanded bioreactor as is illustrated in the lower portion of the bioreactor in FIG. 1.

The inlet 14 and outlet 15 are in fluid communication with the containment portion. The inlet 14 and outlet 15 are horizontally opposed to one another with the inlet 14 being smaller in diameter than the outlet 15.

The upper end cap 16 is attached to the outer wall 20 in a manner similar to the lower end cap 13.

The upper end cap 16 attaches to the inner wall 21 of the containment portion. The main opening 17 in the upper end cap 16 for the rigid core 11 is located centrally with a secondary opening 24 provided for a bleed/pressure relief valve 25. The secondary opening 24 is threaded to provide a removable attachment means for the bleed/pressure relief valve 25.

The main opening 17 has an associated collar 26 to attach the inner wall 21 of the containment portion thereto. The collar 26 is annular to receive the rigid core 11 and then extend through the main opening 17 in the upper end cap 16 which will be suitably dimensioned to receive both the rigid core 11 and the collar 26. The collar 26 has an outer seating portion to locate the collar 26 on the upper end cap 16 and be engaged there. An O-ring sealing means 27 is also provided on the collar 26 in an internal seating groove to form a fluid tight seal with the outer surface of the rigid core 11. The lower portion of the collar 26 is provided with an internally threaded portion engageable with the externally threaded portion on the inner wall 21 of the containment portion.

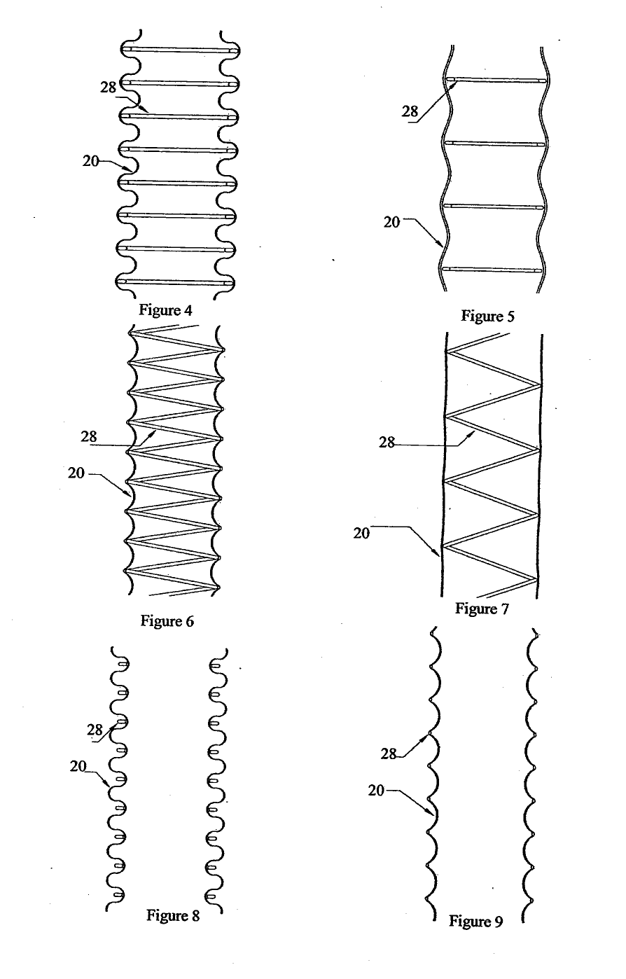

Various configurations and numbers of reinforcement members 28 used according to preferred embodiments are illustrated in FIGS. 4 to 9. For example, according to the embodiment illustrated in FIGS. 4 and 5, a plurality of annular or ring-shaped reinforcement members 28 are provided, spaced over the height of the outer wall 20 with a portion of the reinforcing member supporting the outer wall 20.

The reinforcement members 28 can be provided either on the inside of the outer wall or outside of the outer wall as illustrated in FIG. 8. Where the reinforcement members 28 are provided internally, the reinforcement members are provided in a portion of the outer wall 20 which bulges outwardly (as illustrated in FIG. 4 in particular), and where the reinforcement members 28 are provided externally of the outer wall 20, the ring members will typically be provided in portions of the outer wall 20 between outward bulges.

In the embodiment illustrated in FIGS. 6 and 7, a helical reinforcement member 28 is provided within the outer wall 20.

In the further embodiment illustrated in FIG. 9, the reinforcement members 28 can be integrated into the outer wall 20 and formed therewith. In this form, the outer wall 20 is manufactured by extrusion of a particular thickness of wall portion and then stretching a portion of the wall down to decreased wall thickness leaving thickened reinforcement portions 28 interspersed with the thinner wall portions.

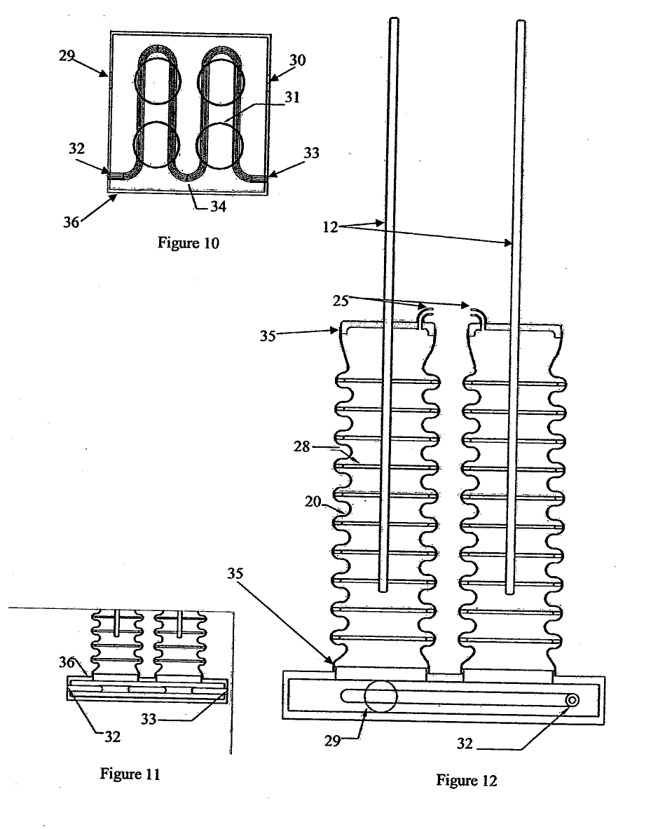

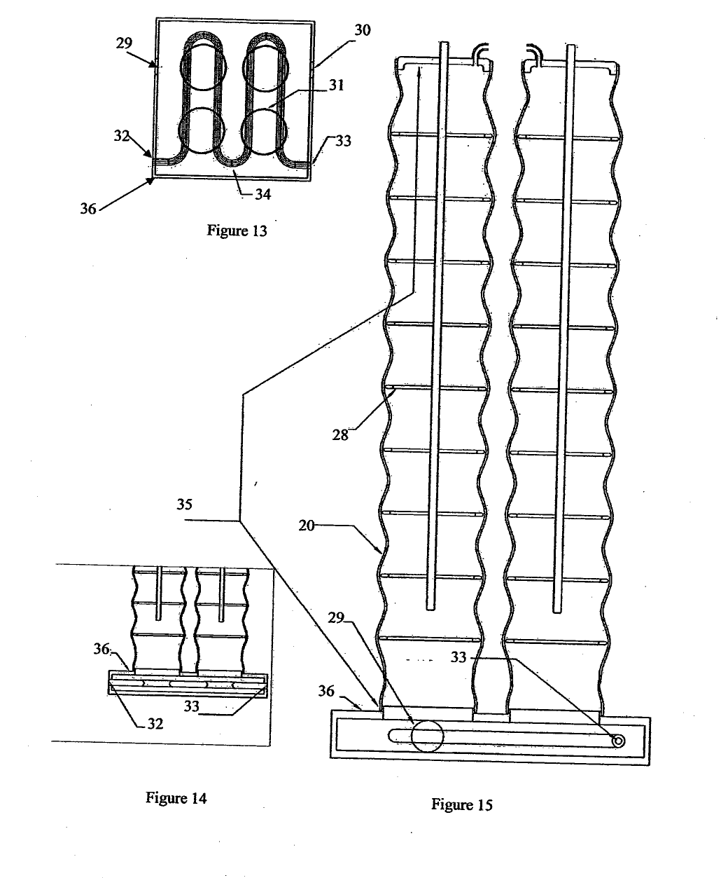

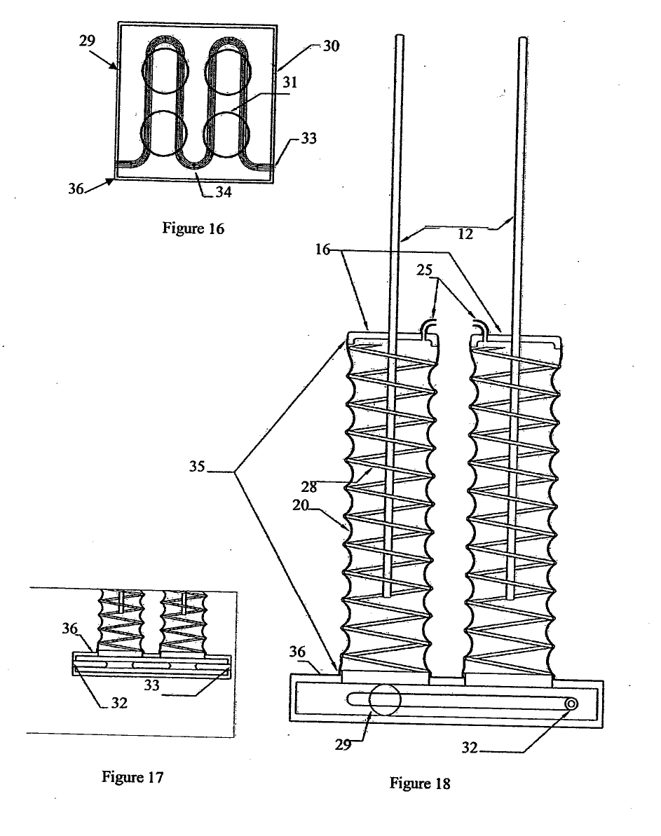

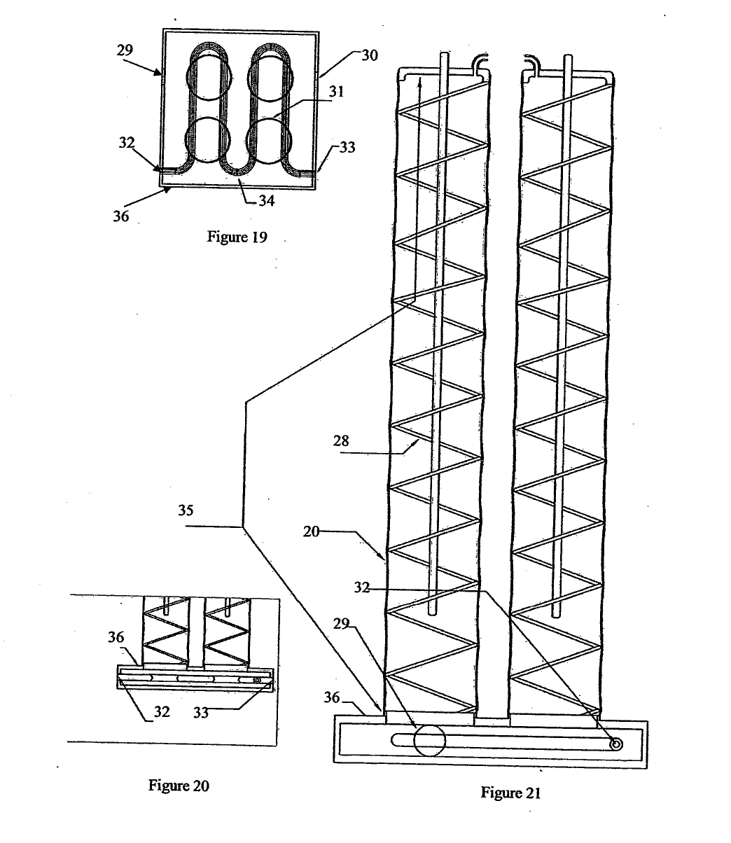

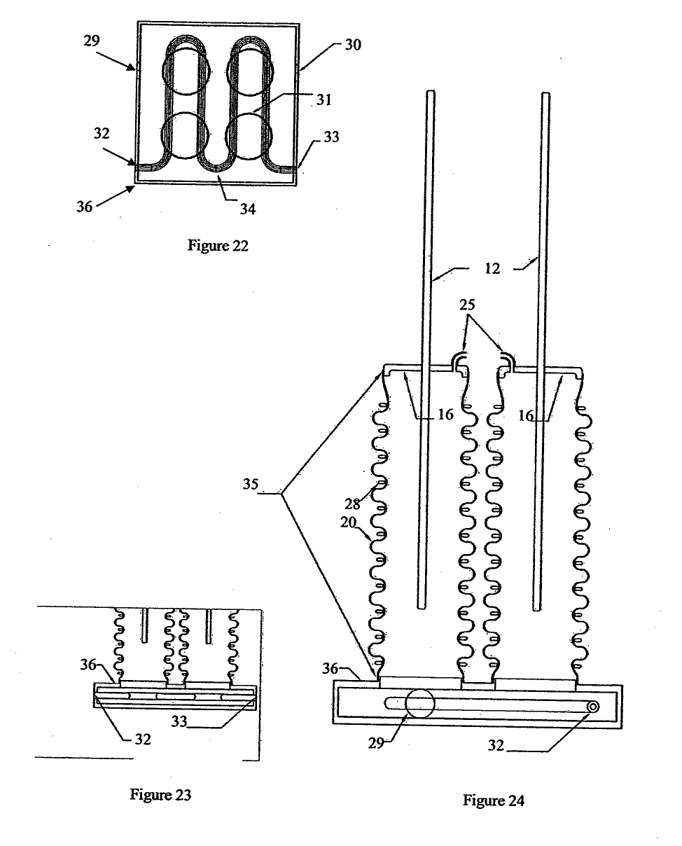

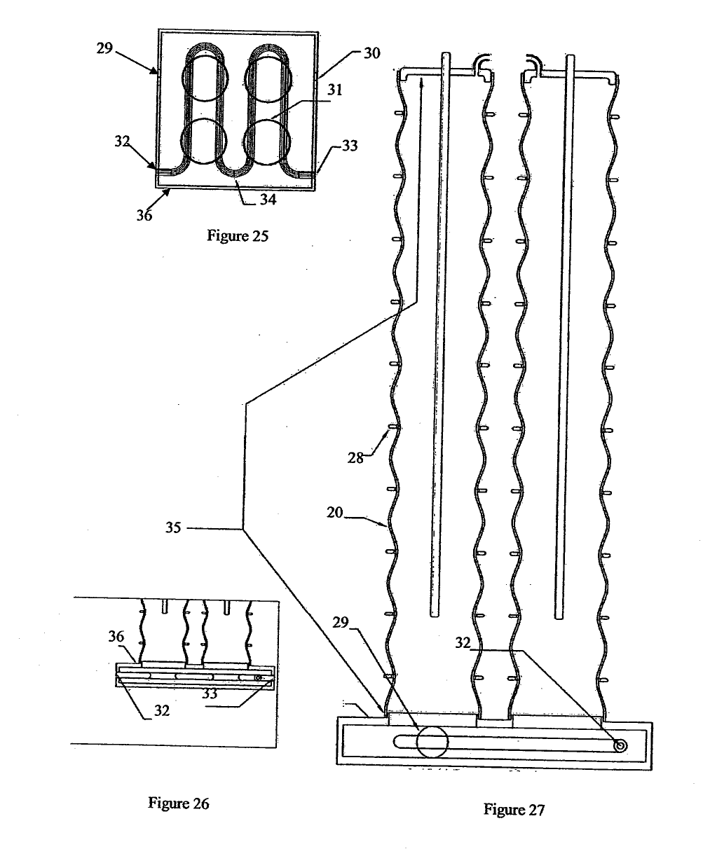

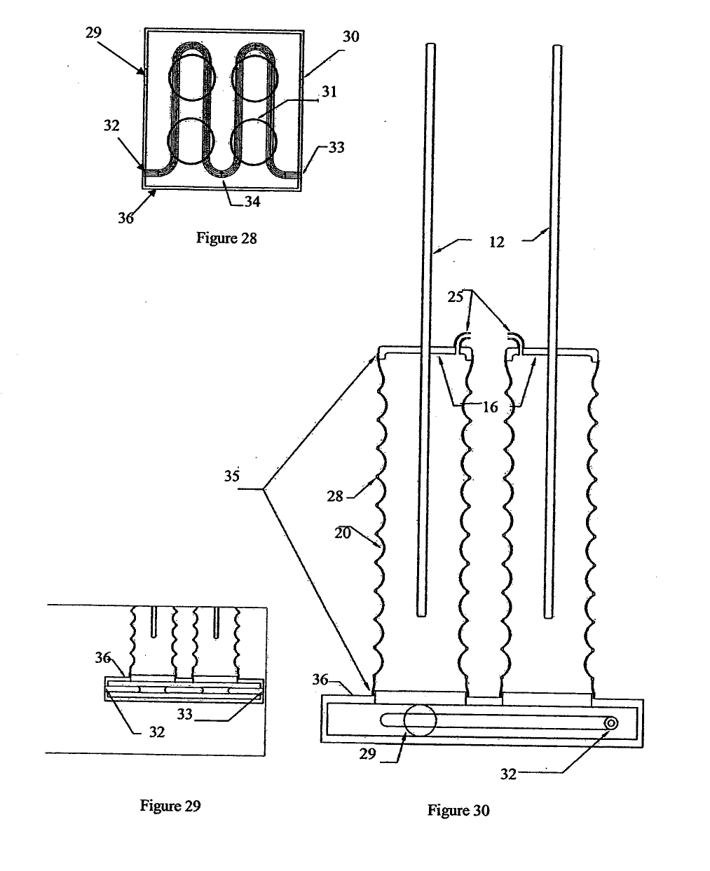

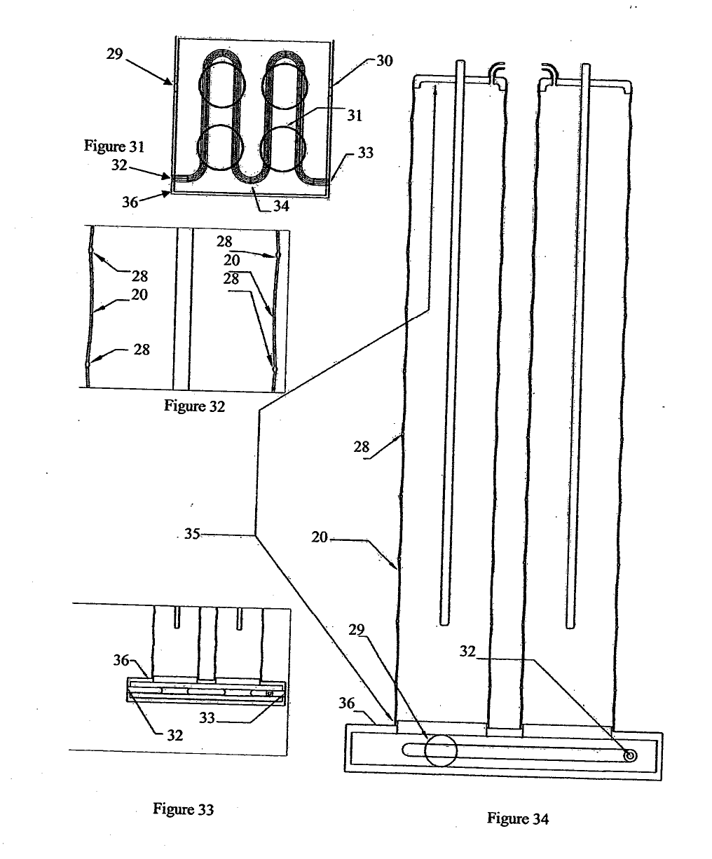

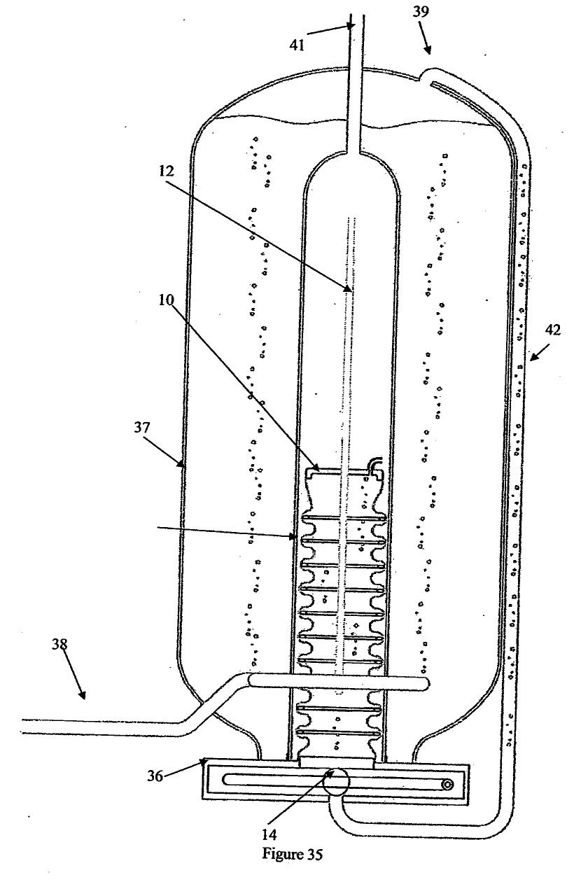

As illustrated in FIGS. 10 to 34, a plurality of bioreactors 10 can be provided mounted on a base member 36. Each base member 36 is provided with utilities such as a heating conduit 34 and a biological material inlet 29 and outlet 30 with appropriate connections to each bioreactor 10. Each bioreactor 10 is typically attached to the base member 36 through the provision of a base fitting 31 and attaching the outer wall 20 of the bioreactor 10 to the base fitting 31. Typically, the connection between the base fitting 31 and the bioreactor 10 will be formed using a clamping means 35.

The conduit 34 for the heating medium will have an inlet 32 and an outlet 33 and be in communication with each of the bioreactors 10 of the base member.

Each base member is configured so that more than one base member 36 and can be located adjacent one another to create a modular assembly and the heating medium inlet 32 and outlet 33 as well as the biological material inlet 29 and outlet 30 of adjacent base members 36 are aligned or communicate. This will allow a single pump means (not shown) to move heating medium through all of the base members 36 and another single pump means to move on biological material through all of the base members 36.

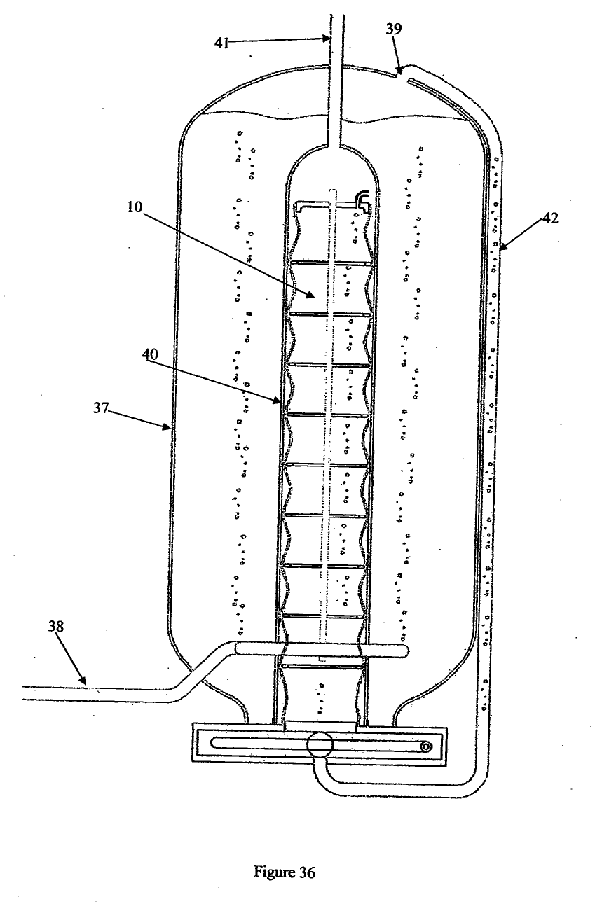

According to an alternative embodiment illustrated in FIGS. 35 and 36, there may be a secondary chamber 37 provided surrounding the bioreactor 10 of the invention. The secondary chamber 37 is a process vessel and is a bioreactor.

According to the illustrated embodiment, the secondary chamber 37 is a secondary bioreactor to treat the incoming material to remove the same or different pollutants as the inner, primary bioreactor 10. The inlet 38 will preferably be into the secondary chamber 37 towards a lower portion of the secondary chamber 37. Located at or towards an upper portion of the secondary chamber 37 is an outlet 39 which is in turn connected to a lower inlet 14 into the primary bioreactor 10 by a transfer pipe 42.

The primary bioreactor 10 and secondary bioreactor chamber 37 are separated by a rigid separating wall 40. The separating wall 40 has a substantially centrally located outlet 41 which according to a illustrated embodiment, is coaxially mounted with the light source.

In the present specification and claims (if any), the word “comprising” and its derivatives including “comprises” and “comprise” include each of the stated integers but does not exclude the inclusion of one or more further integers. Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, or characteristics may be combined in any suitable manner in one or more combinations.

1. A variable volume bioreactor including a rigid core associated with a light source, an outer expandable growth containment portion located concentrically about the rigid core, a lower end portion associated with an inlet to supply culture medium and an outlet, and an upper end cap with an opening therein through which the rigid core passes, wherein the outer containment portion expands as the cellular biological material contained in said portion grows and expands.

2. A variable volume bioreactor according to claim 1 of a cylindrical shape with the outer containment portion and light source located concentrically about a central axis.

3. A variable volume bioreactor according to claim 1 or claim 2 wherein the bioreactor is substantially vertical in orientation and growth of the biological material will force expansion of the containment portion upwardly.

4. A variable volume bioreactor according to any one of the preceding claims including a rigid core of fixed height, associated with the light source.

5. A variable volume bioreactor according to any one of the preceding claims wherein the outer expandable growth containment portion includes an outer wall and an inner wall with the containment portion defined therebetween.

6. A variable volume bioreactor according to claim 5 wherein the inner wall of the containment portion is translucent and has a concertina-like configuration allowing length adjustment.

7. A variable volume bioreactor according to claim 5 or claim 6 wherein the outer wall of the containment portion is opaque and has a concertina-like configuration allowing length adjustment.

8. A variable volume bioreactor according to claim 6 or claim 7 wherein each of the concertina-like walls are formed of a flexible material with a plurality of hinge lines extending circumferentially about each wall to allow expansion and contraction.

9. A variable volume bioreactor according to any one of claims 5 to 8 wherein including a moveable upper end cap with an opening therein through which the light source passes, the opening having an associated collar to attach the inner wall of the containment portion thereto, the collar provided with an outer seating portion to locate the collar on the end cap and be engaged there.

10. A variable volume bioreactor according to any one of claims 1 to 4 wherein the containment portion is provided between an outer containment wall and the light source, without an inner wall.

11. A variable volume bioreactor according to claim 10 including a movable upper end cap having an opening to receive the light source and provided with a sealing grommet associated with the opening, the end cap slidable upwardly and downwardly relative to the light source.

12. A variable volume bioreactor according to any one of the preceding claims including an inlet and outlet in fluid communication with the containment portion, the inlet and outlet are typically horizontally opposed to one another.

13. A variable volume bioreactor according to any one of the preceding claims including a moveable upper end cap with an opening therein through which the light source passes.

14. A variable volume bioreactor according to any one of the preceding claims wherein at least one reinforcement member is provided, spaced over the height of the outer containment portion with a portion of the member supporting the outer containment portion.

15. A variable volume bioreactor according to claim 14 wherein a plurality of annular reinforcement members are provided on the outside of the outer containment portion.

16. A variable volume bioreactor according to claim 14, wherein a helical reinforcement member is provided on the outer containment portion.

17. A variable volume bioreactor system including an inner bioreactor according to claim 1 and wherein a secondary bioreactor chamber is provided surrounding the inner bioreactor.

18. A variable volume bioreactor system according to claim 17 including an inlet provided into the secondary chamber, an outlet from the secondary bioreactor chamber connected to a lower inlet into the inner bioreactor.

19. A variable volume bioreactor system according to claim 17 or claim 18 wherein the inner bioreactor and secondary bioreactor chamber are separated by an outer containment portion of the inner bioreactor.

20. A variable volume bioreactor system according to claim 19 wherein the outer containment portion will normally has a substantially centrally located outlet at an upper region which is coaxially mounted with the light source.

21. A variable volume bioreactor system wherein a plurality of bioreactors according to claim 1 are provided mounted on a base member.

22. A variable volume bioreactor system according to claim 21 wherein the base member is provided with utilities connections and a biological material inlet and outlet with appropriate connections to each bioreactor.

23. A variable volume bioreactor system according to claim 18 wherein each base member is configured so that more than one base member can be located adjacent one another and the utilities connections and the biological material inlet and outlet of adjacent base members are in communicate.
